(19) United States
(12) Patent Application Publication
(10) Pub. No.: US 2026/0060552 A1
(43) Pub. Date: Mar. 5, 2026

(54) Protein Detection Device and Protein Detection Method

(71) Applicants: TOA CORPORATION, Kobe-shi, Hyogo (JP); PROGENICYTE JAPAN CO., LTD., Kobe-shi, Hyogo (JP)

(72) Inventors: Satoshi MIYATA, Kawanishi-shi (JP); Neung SUH, Kakogawa-shi (JP)

(21) Appl. No.: 19/314,198

(22) Filed: Aug. 29, 2025

Related U.S. Application Data
(60) Provisional application No. 63/689,693, filed on Aug. 31, 2024.

(51) Int. Cl. A61B 5/00; A61B 3/10; A61B 5/145; A61B 5/1495

(52) U.S. Cl. CPC A61B 5/0095; A61B 3/10; A61B 5/0022; A61B 5/14546; A61B 5/1495; A61B 5/4064; A61B 5/7246; A61B 5/7267; A61B 5/7282; A61B 5/746; A61B 2503/40

(57) ABSTRACT

A system and method for protein detection are provided, configured to non-invasively identify proteins exhibiting specific structural conformations within a biological target. The system comprises a light source operable to irradiate the target at a predetermined pulse cycle, a sound detection device configured to capture acoustic signals generated via the photoacoustic effect, and an information processing unit that analyzes the detected acoustic signals to determine the presence or accumulation of a target protein. This technique facilitates early-stage detection of disease-associated proteins, such as amyloid-β and misfolded α-synuclein fibrils, without requiring complex imaging modalities or invasive biopsy procedures.

### CROSS-REFERENCE TO RELATED APPLICATIONS

This application claims the benefit of U.S. Provisional Application No. 63/689,693, filed on Aug. 31, 2024, the entire contents of which are incorporated herein by reference.

### BACKGROUND

### Field of the Invention

The present disclosure relates to a detection apparatus and a detection method for detecting a protein having a predetermined structure in a subject.

### Description of Related Art

Early detection of diseases is highly desirable, as timely intervention can improve clinical outcomes and quality of life. However, conventional diagnostic techniques often rely on invasive biopsy sampling or complex imaging technologies, which can be burdensome for both patients and clinicians, particularly during the early stages of disease. Therefore, there is a strong need for simpler, less expensive, and minimally invasive methods to detect disease-associated proteins at an early stage.

Detecting proteins with specific structural characteristics within organs and tissues of animals, including humans, has been shown to be effective in assessing the presence and progression of various diseases. For example, the detection of amyloid fibrils in the retina may provide insights not only into Alzheimer's disease (AD), but also into other conditions involving abnormal protein aggregation.

Photoacoustic imaging is one known method for detecting substances within biological tissues. In this technique, light is irradiated onto the tissue, and the acoustic signal (ultrasound) generated from the target substance is detected to visualize the target. However, its sensitivity is limited because it typically requires a substantial mass of the target substance to generate a detectable signal.

### SUMMARY OF THE INVENTION

Conventional photoacoustic imaging may fail to detect a target substance unless it is present in relatively large quantities within biological tissue. That is, the sensitivity of photoacoustic imaging is limited; if the accumulation of the target substance is insufficient, it may not be visualized as an image. Consequently, detection of proteins with specific structural characteristics using photoacoustic imaging may only become feasible at later stages of disease progression, after substantial protein accumulation has occurred, thereby hindering early diagnosis.

Moreover, photoacoustic imaging systems tend to be complex and expensive, requiring specialized components such as high-sensitivity cameras and pulsed laser sources. These technical and cost barriers limit the practical application of photoacoustic imaging for simple, early-stage disease screening. In view of the foregoing, it is an object of the present disclosure to provide a simplified and cost-effective technique capable of detecting proteins contained in a biological subject even when the protein is present in low amounts. To achieve the above object, a protein detection device according to one aspect of the present disclosure comprises:
A light source is configured to emit light pulses directed toward a biological target at a predetermined irradiation cycle, which refers to the number of light pulses per minute;
A sound detection device is positioned in proximity to the target and configured to detect acoustic signals generated via the photoacoustic effect when pulsed light is absorbed by the tissue; and
An information processing device configured to determine the presence of a protein having a predetermined structural configuration in the target object based on the magnitude of the detected sound.

In another aspect, a protein detection method comprises irradiating a target object with light at a predetermined irradiation cycle, detecting photoacoustically generated sound using a sound detection device, and determining the presence of a protein with a predetermined structure based on the magnitude of the detected sound. The method may be implemented using the device described in the first aspect of the invention and is particularly well-suited for detecting misfolded or aggregated proteins such as amyloid-β or α-synuclein fibrils, which are associated with AD and Parkinson's disease (PD), respectively.

The inventors of the present disclosure discovered that when light is irradiated onto a target object at a predetermined cycle, proteins with specific structural characteristics contained in the target emit detectable sound via the photoacoustic effect. Notably, even at low contents, these proteins generate acoustic signals that can be captured by the sound detection device. Because the acoustic signal acquisition can be implemented with simple instrumentation, the proposed device and method enable early-stage detection of disease-associated proteins in a non-invasive, cost-effective manner. This facilitates the early diagnosis of diseases associated with such proteins and improves the feasibility of routine clinical screening.

### BRIEF DESCRIPTION OF THE DRAWINGS

FIG. 1 is a schematic diagram illustrating a protein detection device according to one embodiment of the present disclosure. The system may include a light source, an acoustic detection unit, and an information processing module configured to, or otherwise capable of, detect proteins based at least part on photoacoustic response.

FIG. 2 is a flowchart illustrating a protein detection method according to one embodiment of the present disclosure. The method may include steps of irradiating a sample with light, detecting sound acoustic signals, and determining the potential presence of proteins based on characteristic of the detected sound.

FIG. 3 is a conceptual flow diagram illustrating calibration strategies for photoacoustic-based protein detection. Examples include absolute calibration with reference samples, relative calibration for longitudinal monitoring, and adaptive thresholding approaches such as AI-based analysis.

FIG. 4 shows example experimental results, in which brain tissue slices of human amyloid-expressing transgenic mice exhibited stronger acoustic responses, within certain frequency ranges compared to slices from wild-type mice. These data are provided for illustrative purposes and demonstrate one embodiment of the technology's applicability to detecting one or more target protein-containing structures.

FIG. 5 presents preliminary findings from a study using human brain tissue. In one example, sections from patient with Alzheimer's disease displayed different photoacoustic responses relative to control subjects. These results are provided as illustrative data supporting potential translational use in clinical or research diagnostics.

### DETAILED DESCRIPTION OF THE INVENTION

Hereinafter, embodiments will be described in detail with reference to the accompanying drawings. However, to avoid redundancy and promote clarity, well-known or repetitive aspects may be omitted. The drawings and following descriptions serve to aid those skilled in the art in fully understanding the invention and are not intended to limit the claims.

### Overview

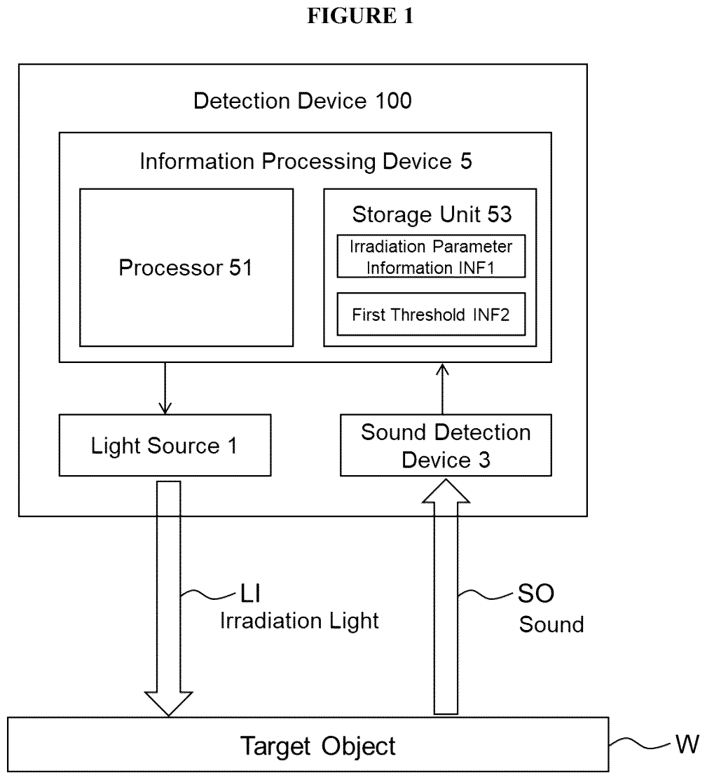

As shown in FIG. 1, the protein detection device 100 irradiates light at a predetermined irradiation cycle onto a target object W, such as animal tissue, to determine the presence or absence of proteins having a predetermined structure. Acoustic signals generated from proteins due to the photoacoustic effect are detected by a sound detection device 3. Based on the magnitude of the detected sound, the presence of the target protein is determined by an information processing device 5.

Detectable proteins include, but are not limited to, amyloid fibers formed from misfolded proteins such as amyloid-β or synuclein, which are often implicated in neurodegenerative diseases, such as AD and PD. When detecting amyloid in the retina, light may be externally irradiated into the eye or directed onto extracted tissues.

The retina is a highly suitable anatomical site for noninvasive detection of disease-associated proteins, such as amyloid-β, due to its structural and physiological characteristics. As an extension of the central nervous system (CNS), the retina shares many biochemical and pathological features with the brain, including the early accumulation of misfolded proteins implicated in neurodegenerative diseases like AD and PD.

Importantly, the retina is optically accessible via the pupil, allowing light-based diagnostic modalities—such as the photoacoustic-based system described herein—to irradiate and analyze retinal tissue without surgical intervention. This noninvasive access makes the retina an ideal target for routine, repeated assessments in clinical and at-home settings. Additionally, studies have demonstrated that amyloid-β deposits can be found in the retinal layers of individuals with early-stage AD, making the retina a potential window into brain pathology.

By directing pulsed illumination (e.g., from a white LED) through the cornea and lens, and detecting photoacoustic signals emitted from retinal proteins via a sensitive microphone, the described system can evaluate protein accumulation without requiring invasive sampling or complex imaging. The retina's thin, layered structure and rich vasculature also enhance the generation of detectable acoustic responses, further supporting its use as a reliable, noninvasive biomarker site.

This method allows simple, early-stage detection of disease-associated proteins with minimal equipment and processing time, potentially allowing for disease monitoring or progression tracking over time through repeated use.

### Configuration

FIG. 1 is a schematic block diagram illustrating a protein detection device 100 according to one embodiment of the present disclosure. As illustrated in FIG. 1, the detection device 100 includes a light source 1, a sound detection device 3, and an information processing device 5:

The light source 1 is configured to emit illumination light LI at a predetermined irradiation cycle. In certain embodiments, the light source 1 is implemented as a white-light LED or a pulsed xenon lamp, which provide, capable of generating stroboscopic pulses at a repetition rate between 10,000 and 20,000 revolutions per minute (rpm). The use of low-power illumination, such as white LEDs or pulsed xenon lamps, reduces energy consumption, eliminates the need for active cooling, and enables safe, portable, and cost-effective protein detection. A target object W (e.g., tissue containing a protein of interest, etc.) is placed in a position to receive light irradiation from the light source 1. This configuration facilitates robust photoacoustic signal generation without requiring fine-tuned wavelength selection. The irradiation cycle may be controlled by the information processing device 5. The use of white light, with its broad spectral range (typically 400-700 nm), enables effective excitation of various target proteins without the need for wavelength-specific optimization.

The sound detection device 3, which may be implemented as a microphone, is positioned to detect acoustic signals (sound SO) generated from the target object W via the photoacoustic effect. This effect arises when proteins with specific structural conformations—such as amyloid fibrils—absorb pulsed light and emit pressure waves. The microphone may be a compact and cost-effective device capable of capturing such acoustic signals with sufficient sensitivity for analysis.

The information processing device 5 comprises a processor 51 and a storage unit 53 and is configured to control the overall operation of the system. The processor 51 controls the light source 1 to emit pulses at a predetermined cycle (e.g., 10,000-20,000 rpm, preferably 12,000-15,000 rpm), thereby inducing acoustic signals in the target object within a detectable frequency range (e.g., 1,500-4,000 Hz). This frequency range falls within the audible spectrum and below the threshold of visual discomfort, ensuring safety and effective signal detection. The processor 51 analyzes the acoustic signals captured by the sound detection device 3 and determines the presence or accumulation of the target protein by comparing the sound magnitude against a predefined threshold. Repeated measurements can indicate disease progression. The storage unit 53 retains relevant system parameters, including irradiation settings (INF1) and detection thresholds (INF2), such as the first threshold value used for protein detection.

The described configuration enables compact, low-cost, and non-invasive protein detection suitable for portable or wearable applications, such as smart glasses or head-mounted displays (HMDs). Owing to the small footprint of components like LEDs and microphones, the system can be seamlessly integrated into wearable platforms. A mobile device, such as a smartphone or tablet, may function as the processing unit, communicating with the wearable via Bluetooth or a wired connection.

### Detection Operation

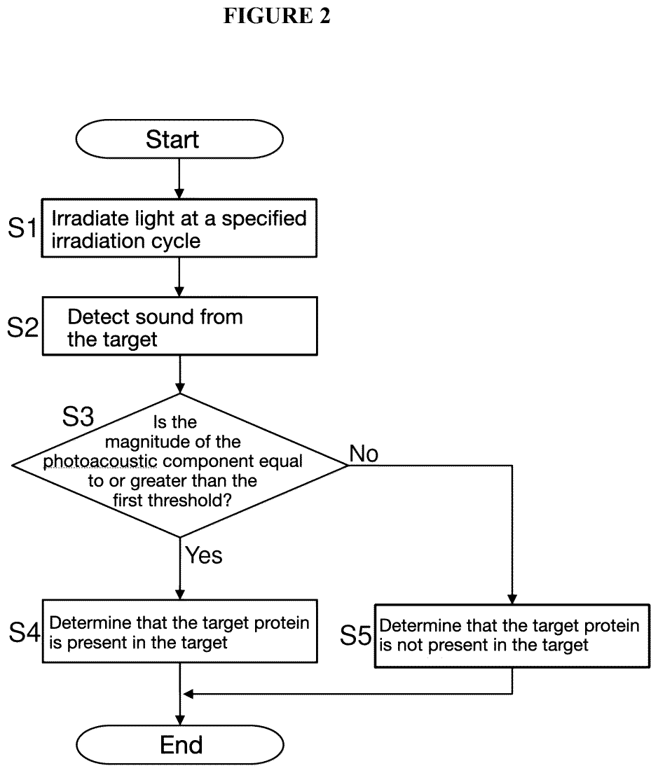

FIG. 2 is a flowchart illustrating an example of a detection method executed by the protein detection device 100.

Step S1: The processor 51 controls the light source 1 to emit pulsed illumination light LI at the specified irradiation cycle retrieved from irradiation parameter information INF1. The light is directed toward the target object W, which may be in vivo tissue (e.g., retina) or ex vivo samples.

Step S2: The sound detection device 3 detects sound SO generated by the target object W in response to the light irradiation. The sound signal is sent to the processor 51.

Step S3: The processor 51 analyzes the detected sound SO to determine whether a frequency component within a predefined range (e.g., 1,500 Hz to 4,000 Hz) exceeds a first threshold value INF2 stored in the storage unit 53. This may involve spectrum decomposition using FFT (Fast Fourier Transform).

Step S4: If the sound magnitude meets or exceeds the threshold (“Yes” in Step S3), the processor 51 determines that a protein having a specific structure is present in the target object W. Optionally, a notification (light or sound) is issued to indicate successful detection. Protein quantity may also be calculated and displayed.

Step S5: If the sound magnitude is below the threshold (“No” in Step S3), the system determines that the target protein is not present or below detectable levels. A different notification may be issued to inform the user.

Various modifications may be made without departing from the essence of the present invention. For example, the procedural steps as set forth above may be reordered or executed in parallel. Signal processing can be implemented through hardware components such as analog bandpass filters. The information processing device may also be cloud-based, allowing for remote storage and analysis of detection data. If a specific wavelength proves more effective for certain targets, the light source or optical filters may be adapted accordingly. Furthermore, additional components, such as user control buttons, may be incorporated to enhance operability. Additionally, the amount of protein may be estimated by correlating sound magnitude to protein concentration using pre-established calibration data.

### Calibration and Comparative Analysis

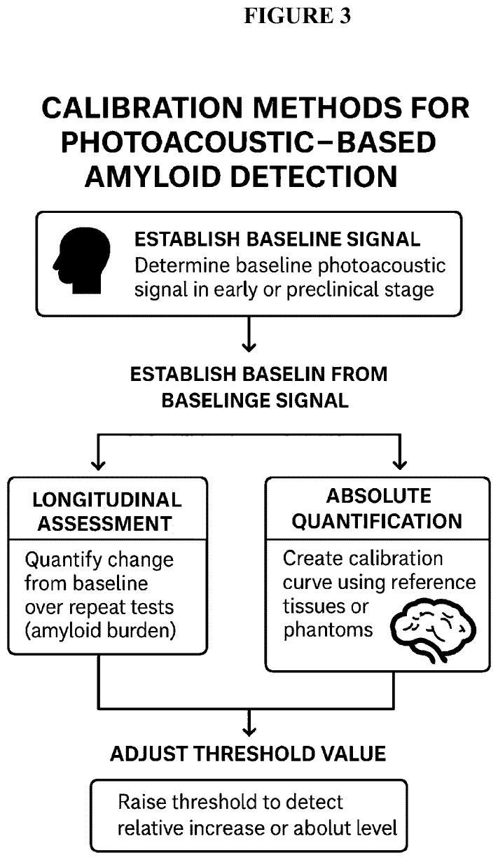

FIG. 3 is a flowchart illustrating two complementary calibration strategies used to enhance the diagnostic utility and reliability of protein detection using a photoacoustic-based system. The top node represents the establishment of a baseline acoustic signal from the target subject, preferably obtained during an early or preclinical disease stage. This baseline can serve in two distinct calibration strategies:

Absolute Calibration: The detected signal is compared to reference curves generated from known samples (e.g., wild-type or transgenic brain tissue, or synthetic phantoms) to estimate the absolute burden of disease-associated proteins such as amyloid-β fibrils. This enables cross-subject comparisons and standardization of diagnostic thresholds.

Longitudinal Calibration: Acoustic signals collected over time from the same subject can be compared against that individual's baseline profile, enabling personalized monitoring of protein accumulation and disease progression. Periodic assessments (e.g., monthly) allow for tracking changes in signal magnitude or spectral characteristics. By analyzing trends in the acoustic data—whether increasing, stable, or decreasing—clinicians can assess the trajectory of pathological protein burden, such as amyloid accumulation. This approach also permits inter-subject comparisons when standardized reference values or calibration data are available.

The lower node indicates that threshold values used to determine protein presence or severity may be dynamically adjusted based on these calibration strategies. Optionally, Artificial Intelligence Calibration trained on large-scale datasets of acoustic signatures and clinical metadata (e.g., age, sex, APOE genotype, cognitive scores) may be employed to dynamically adjust detection thresholds and improve predictive accuracy.

### Experimental Example 1: Mouse Brain Slice Validation of Amyloid Detection

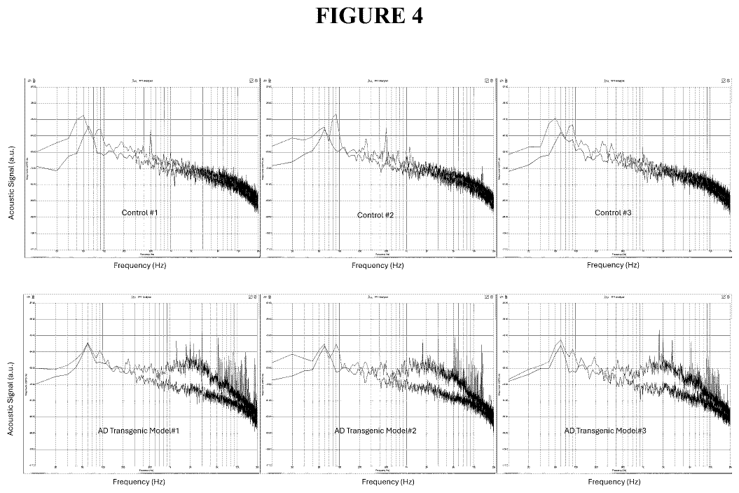

FIG. 4 illustrates representative acoustic signal data obtained from an experimental validation study using ex vivo brain slices from wild-type (WT) mice and amyloid-β (Aβ) plaque-bearing APP transgenic mice (5xFAD strain, B6SJL-Tg(APPSwFlLon, PSEN1M146LL286V)6799Vas/Mmjax). These transgenic mice harbor human APP mutations (Swedish, Florida, London) and PSEN1 mutations (M146L, L286V), which induce early and robust Aβ plaque formation beginning at approximately 3 months of age.

Frozen coronal brain sections (40μm thickness) from 8-month-old mice were mounted on glass slides and placed on acoustically dampened Styrofoam substrates. Illumination was applied using a digital stroboscope equipped with a white LED light source pulsing at approximately 12,000 rpm. A MEMS (Micro Electro Mechanical System) microphone, positioned to avoid direct light exposure, captured the acoustic signals generated by the photoacoustic effect. Control microphones were similarly placed without tissue or light exposure to establish background baselines.

The figure presents six frequency-domain plots: the top row shows fast Fourier transform (FFT) spectra from three WT mice (Control #1-3), and the bottom row from three transgenic AD model mice (AD Model #1-3). In WT samples, low and relatively flat signal amplitudes were observed across the frequency spectrum. In contrast, AD slices exhibited distinct elevations in the 2,000-3,500 Hz range—highlighted by blue and yellow traces—indicating the presence of amyloid-specific acoustic signatures exceeding the background noise threshold.

Both WT and AD slices generated measurable acoustic responses upon light irradiation. Notably, AD slices consistently exhibited greater signal amplitudes compared to WT controls, particularly in the 2,000-3,500 Hz frequency range. The most pronounced differences between the two groups were observed when the light source operated at pulse cycles between 12,000 and 15,000 rpm. Importantly, since the only variable distinguishing the WT and AD samples was the presence of Aβ plaques, these findings support the specificity of the detected acoustic signals to amyloid accumulation.

Data acquisition was conducted under normal laboratory environmental conditions without specialized acoustic isolation. Despite ambient noise from air handling systems, equipment fans, and human activity, the detection system consistently identified Aβ-related acoustic signatures with signal levels above the defined threshold. This underscores the robustness and real-world applicability of the proposed system for non-invasive protein detection.

To further enhance signal-to-noise ratio (SNR), the information processing unit implements frequency-domain filtering, isolating periodic signals in the 2,000-3,500 Hz band while attenuating non-specific background noise. These results validate the practicality of the disclosed apparatus and method for detecting pathological protein accumulation under ambient conditions.

### Experimental Example 2: Human Brain Slice Analysis

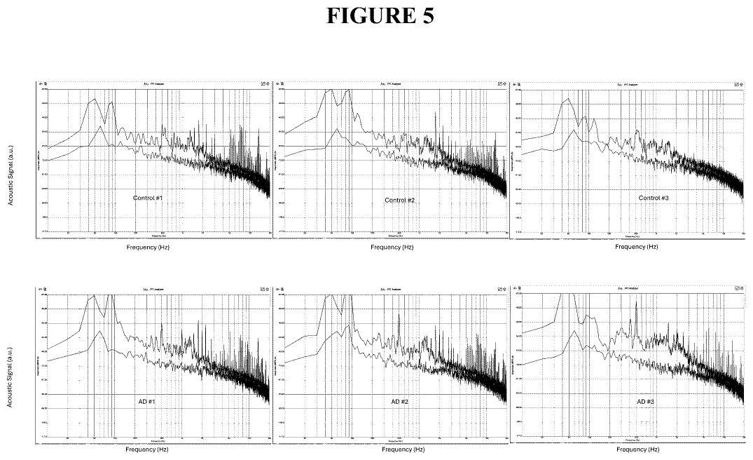

FIG. 5 presents representative acoustic signal frequency spectra obtained from 40 μm cryosectioned human temporal lobe tissues of AD patients and cognitively normal control subjects aged 65+, using the photoacoustic detection apparatus described herein.

Each plot illustrates the acoustic signal intensity (in arbitrary units) as a function of frequency. The upper panel includes data from three control subjects (Control #1-3), while the lower panel shows results from three AD patients (AD #1-3).

Among the control samples, Control #3 exhibited a spectral profile notably different from the AD samples, characterized by an absence of elevated acoustic signals in the 2,000-3,500 Hz range. In contrast, Control #1 and Control #2 displayed frequency profiles comparable to those of AD samples, suggesting the presence of early-stage or preclinical amyloid-β (Aβ) accumulation. This observation is consistent with prior reports indicating that Aβ deposition can occur up to 15 years before clinical symptom onset.

All human brain sections, both control and AD, exhibited baseline low-frequency acoustic signals, which are likely attributable to common structural components present in cortical tissue. However, only the AD and potentially preclinical control samples demonstrated elevated signal intensity within the 2,000-3,500 Hz range, corresponding to the acoustic signature associated with Aβ plaques as identified in prior mouse experiments.

These findings underscore the sensitivity of the disclosed photoacoustic detection system in distinguishing Aβ-positive from Aβ-negative tissue. Moreover, they support the potential clinical utility of the system in detecting early, asymptomatic Aβ accumulation in human brain tissue, thereby facilitating non-invasive or minimally invasive early diagnosis of AD.

As described above, the present invention encompasses both a novel apparatus and an associated method for detecting proteins with specific structural characteristics, such as amyloid-β fibrils, using a non-invasive photoacoustic-based approach. Detectable target proteins include, but are not limited to, amyloid fibers derived from misfolded proteins such as amyloid-β and α-synuclein, commonly associated with neurodegenerative diseases like AD and PD.

When targeting amyloid in the retina, for example, light may be externally applied to the eye or directed toward extracted retinal tissues.

Claim 1 defines a protein detection device comprising a light source, a sound detection component, and an information processing unit configured to determine the presence or accumulation of a target protein based on the acoustic response elicited by pulsed light irradiation (see FIG. 1).

Claim 16 defines a corresponding method of using the device described in Claim 1, detailing the steps of irradiating light onto a biological subject, detecting resulting acoustic signals, and determining the presence or level of protein accumulation based on the intensity and frequency of the detected sound (see FIG. 2).

The method of Claim 16 is intended to be implemented using the apparatus described in Claim 1 and its dependent claims. The inclusion of method claims serves to broaden the scope of protection, ensuring that both the system architecture and its functional use are encompassed within the scope of the present invention.

### Technical Advantages Over Prior Art

Unlike the prior art, such as JP2023-060587 A, which is primarily directed toward photoacoustic imaging of tissue to visualize pathological changes, the present invention provides a non-imaging, signal-based detection system for proteins such as amyloid-β. This fundamental distinction offers the following technical advantages:

Elimination of Image Reconstruction: The prior art requires multiple photoacoustic signals from spatially resolved coordinates to generate an image using reconstruction algorithms and high-resolution transducers. In contrast, the present invention relies solely on the magnitude and frequency profile of a single-point acoustic signal, removing the need for complex spatial scanning, synchronization, or tomographic processing.

Reduced Hardware Requirements: Since the present invention does not require imaging, it can avoid the use of ultrasonic detector arrays or scanning actuators, high-speed analog-to-digital converters for spatial resolution, and complex software frameworks for image formation such as beamforming or delay-and-sum algorithms. As a result, the system may be implemented with at least one acoustic sensor and at least one light source (e.g., an LED or other light source capable of generating suitable excitation), thereby enabling a simplified and lower-cost configuration.

Faster Acquisition and Interpretation: The simplified acoustic signature analysis (e.g., FFT of a short signal) enables near-real-time assessment of protein accumulation without waiting for image acquisition or reconstruction. This supports point-of-care, rapid screening applications.

Compact, Wearable, and Mobile-Compatible: The design enables integration into mobile or wearable platforms, such as a smartphone accessory or glasses-mounted system—impractical with conventional imaging systems due to size, power, and data processing demands.

Reduced Spatial Targeting Requirement: The disclosed system detects proteins via overall signal changes in the region of interest, such as the retina, without requiring micrometer-level localization or depth discrimination. In contrast, the prior art requires accurate targeting and image alignment for meaningful interpretation.

Broader Access and Deployment: Imaging systems are typically used in specialized clinical settings due to their cost and complexity. In contrast, the present invention facilitates low-cost mass screening, making it suitable for primary care, home use, or low-resource environments. Furthermore, unlike conventional photoacoustic imaging systems that require controlled or shielded environments, the disclosed detection system operates effectively under ambient noise conditions. The present invention allows for simplified deployment in non-clinical settings without acoustic isolation chambers or costly shielding apparatus, thereby reducing system cost and improving usability.

What is claimed is:

1. A protein detection device comprising:
a light source configured to irradiate a target object with light at a predetermined irradiation cycle;
a sound detection device configured to detect sound generated from the target object due to a photoacoustic effect upon irradiation; and
an information processing device configured to determine the presence of a disease-related protein based on a magnitude of the detected sound, wherein the information processing device comprises a non-transitory memory storing a classification algorithm trained to identify disease-related protein signatures from sound signal features.

2. The device of claim 1, wherein the information processing device determines that the protein is present when the detected sound magnitude is equal to or greater than a predetermined threshold stored in the non-transitory memory.

3. The device of claim 1, wherein the information processing device compares a magnitude of the detected sound to previously recorded and time-stamped sound data stored in a database to assess protein accumulation trends in the target object.

4. The device of claim 1, wherein the irradiation cycle is within a range from 10,000 rpm to 20,000 rpm and optimized for acoustic signal amplification.

5. The device of claim 1, wherein the light source emits broadband white light covering wavelengths from 400 nm to 700 nm.

6. The device of claim 1, wherein the sound detection device is a capacitive or piezoelectric microphone with a frequency response covering at least 1,000 Hz to 5,000 Hz.

7. The device of claim 1, wherein the information processing device is further configured to calculate an estimated concentration of the disease-related protein based on amplitude modulation and frequency shift analysis of the detected sound.

8. The device of claim 1, wherein the disease-related protein comprises amyloid fibers, and the information processing device is configured to output a diagnostic alert when amyloid accumulation in the retina exceeds a diagnostic threshold indicative of a neurodegenerative disease.

9. The device of claim 1, wherein the target object comprises human or animal retinal or brain tissue.

10. The device of claim 1, wherein the target object is an animal retina, and the light source is configured to irradiate the retina trans-sclerally or through the pupil.

11. The device of claim 1, wherein the sound detection device is positioned externally with acoustic coupling to the eye and detects sound within a frequency range of 1,500 Hz to 4,000 Hz.

12. The device of claim 1, wherein the information processing device is integrated into a wearable terminal configured for real-time data processing.

13. The device of claim 12, wherein the wearable terminal is selected from the group consisting of a smartphone, smartwatch, tablet, smart glasses, or head-mounted display.

14. The device of claim 12, further comprising a wireless communication module configured to transmit the detected sound data to a cloud-based system for centralized analysis and longitudinal tracking.

15. The device of claim 12, wherein the information processing device displays a comparison between the currently detected sound and a historical trend line for the target object.

16. A method for detecting a disease-related protein in a target object, comprising:
irradiating the target object with light at a predetermined irradiation cycle;
detecting sound emitted from the target object due to a photoacoustic effect;
analyzing the detected sound using a machine learning classifier trained to identify disease-related protein signatures; and
determining the presence of the disease-related protein based on at least one of the magnitude or frequency components of the detected sound.

17. The method of claim 16, further comprising comparing the detected sound magnitude to a predetermined threshold stored in a memory device.

18. The method of claim 16, further comprising comparing the detected sound magnitude to a previously recorded sound magnitude to assess the progression of protein accumulation.

19. The method of claim 16, wherein the disease-related protein comprises amyloid fibers, and wherein the method comprises detecting amyloid fiber accumulation in the retina and outputting a diagnostic flag when accumulation exceeds a threshold indicative of a neurodegenerative condition.
